SEQUENCE LISTING (4 sequences)

SEQ ID NO: 1 - DNA, 22 bp, Artificial Sequence
  ITS1F Primer
cttggtcatttagaggaagtaa

SEQ ID NO: 2 - DNA, 20 bp, Artificial Sequence
  ITS2R Primer
gctgcgttcttcatcgatgc

SEQ ID NO: 3 - DNA, 33 bp, Artificial Sequence
  Forward overhang Primer
tcgtcggcagcgtcagatgtgtataagagacag

SEQ ID NO: 4 - DNA, 34 bp, Artificial Sequence
  Reverse overhang Primer
gtctcgtgggctcggagatgtgtataagagacag

(19) United States
(12) Patent Application Publication
(10) Pub. No.: US 2023/0127277 A1
(43) Pub. Date: Apr. 27, 2023

(54) MICROBIOME-BASED THERAPEUTICS

(71) Applicant: Cornell University, Ithaca, NY (US)

(72) Inventors: Iliyan ILIEV, New York, NY (US); Irina LEONARDI, Long Island City, NY (US)

(21) Appl. No.: 17/907,449

(22) PCT Filed: Mar. 29, 2021
(86) PCT No.: PCT/US2021/024724

§ 371 Date: Sep. 27, 2022

Related U.S. Application Data
(60) Provisional application No. 63/000,709, filed on Mar. 27, 2020.

(51) Int. Cl. C12Q 1/04; A61P 37/00; A61K 35/741

(52) U.S. Cl. CPC C12Q 1/04; A61P 37/00; A61K 35/741

(57) ABSTRACT

Provided herein is a method of treating an individual afflicted with an inflammatory bowel disease utilizing Candida abundance as a biomarker of responsiveness. The method comprises determining levels of Candida in a sample from the gastrointestinal tract of an individual, and if the level of Candida is higher than a reference level, identifying the individual as suitable for microbiota transplantation therapy (MTT), and optionally, administering to such an individual the MTT, and in individuals having gastrointestinal tract Candida levels lower than a reference level, increasing the Candida levels prior to prior to administration of MTT.

### CROSS-REFERENCE TO RELATED APPLICATIONS

This application claims priority to U.S. provisional patent application No. 63/000,709, filed on Mar. 27, 2020, the disclosure of which is incorporated herein by reference.

### STATEMENT REGARDING FEDERALLY FUNDED RESEARCH

This invention was made with government support under Grant Number DK113136 awarded by the National Institute of Diabetes and Digestive and Kidney Diseases. The government has certain rights in the invention.

### BACKGROUND OF THE DISCLOSURE

Fecal microbiota transplantation (FMT) from heterologous donors has been successfully used to treat intestinal Clostridioides difficile infections (CDI). This success has sparked interest in extending its application to other diseases affecting the gastrointestinal tract, such as inflammatory bowel disease (IBD) (Allegretti et al. 2019, The Lancet 394 (10196):420-431. doi: 10.1016/S0140-6736(19)31266-8; Weingarden et al., 2017, Gut Microbes 8 (3):238-252. doi: 10.1080/19490976.2017.1290757; Paramsothy et al. 2017, The Lancet 389 (10075):1218-1228. doi: 10.1016/S0140-6736(17)30182-4), graft versus host disease (GVHD)(Kakihana et al. 2016, Blood 128 (16):2083-2088. doi: 10.1182/blood-2016-05-717652), metabolic syndrome (Vrieze et al., 2012, Gastroenterology 143 (4):913-916.e7. doi: 10.1053/j.gastro.2012.06.031), and immune checkpoint inhibitor associated colitis (ICIAC) (Wang et al., 2018, Nature Medicine 24 (12):1804-1808. doi: 10.1038/s41591-018-0238-9). In contrast to CDI, where FMT successfully treats>90% of cases (van Nood et al., 2013, New England Journal of Medicine 368 (5):407-415. doi: 10.1056/NEJMoa1205037; Quraishi et al., 2017, Alimentary Pharmacology & Therapeutics 46 (5):479-493. doi: 10.1111/apt.14201), FMT application in IBD faces significant challenges due to complex disease etiology and the absence of a clear microbial target. While FMT is reported to promote symptoms resolution (also termed clinical remission) and mucosal healing in ulcerative colitis (UC), a major form of IBD, the benefits were limited to a subset of patients (Paramsothy et al., 2017, The Lancet 389 (10075):1218-1228. doi: 10.1016/S0140-6736(17)30182-4). This heterogeneity highlights the urgent need to identify mechanisms and define predictive markers to forecast FMT treatment response prior to the initiation of therapy.

### SUMMARY OF THE DISCLOSURE

In this disclosure, an unexpected association between intestinal Candida sp. colonization level and positive clinical outcome of FMT for irritable bowel disease (IBD) patients was observed. Based at least in part on these unexpected observations, the disclosure provides a method of identifying individuals afflicted with IBD, who will benefit from microbiota transplantation therapy (MTT). The method comprises determining the level of candida sp. in a sample from the gastrointestinal tract ((GI tract) or may be referred to herein as “gut”) of a patient with IBD, and if the level is the same or higher than a reference level, then identifying the individual to be suitable for MTT treatment. If the level is lower than a reference level, then the individual is considered for a therapy other an MTT, or is considered suitable for a therapy to increase Candida levels in the gut prior to MTT treatment.

In an embodiment, the method comprises: in a sample obtained from the individual's GI tract, determining the level of Candida sp. to be the same or higher than a reference level; and administering to the individual, a therapy comprising microbiota transplantation therapy; or in a sample obtained from the individual's GI tract, determining the level of Candida sp. to be lower than a reference level, administering a composition to the individual to increase the level of Candida sp. such that the Candida sp. levels increase to the same or higher than the reference level, and administering to the individual, a therapy comprising microbiota transplantation therapy.

IBD may be ulcerative colitis or Crohn's disease. In embodiments, the sample from the individual's GI tract may be a stool sample or may be obtained directly from any part of the GI tract, such as via colonoscopy or biopsy. A reference level may be a level of Candida sp. that is present in healthy individuals who do not have IBD or it may be a level of Candida sp. from individuals having IBD who have responded positively to MTT.

The disclosure also provides kits for treating inflammatory bowel disease, the kit comprising one or more reagents for detecting and/or quantitating Candida sp. levels in a sample, e.g., a sample from the gut of an individual; and MTT composition for administration to the individual. The kit may optionally comprise instructions on use of the kit, including reference levels for comparing the Candida levels. The kit may optionally comprise compositions for increasing the level of Candida in the gut.

### BRIEF DESCRIPTION OF THE FIGURES

FIG. 1. Mycobiota composition. A. Alpha diversity measured by observed OTU and Shannon Index for donor, fecal transplantation recipient (FMT), and placebo recipient (CTRL) pre- and post-FMT. B. Ratio between Ascomycota and Basidiomycota are shown for donor, fecal transplantation recipient (FMT), and placebo recipient (CTRL) at pre—and post-FMT. C. Stack plot of relative abundances at the genus level for the 15 genera with the highest average abundance are shown. Dots represent individual patients, line and bars represent mean+/−SEM.

FIG. 2. A. Relative abundance of Candida in fecal transplantation recipient (FMT), and placebo recipient (CTRL) at pre- and post-FMT. The paired mean difference between FMT pre- and post-FMT is 0.0234 [95.0% CI −0.203, 0.266]; P value>0.1 (Wilcoxon test). The paired mean difference between CTRL pre- and post-FMT is 0.111 [95.0% CI −0.198, 0.371]; pValue>0.1 (Wilcoxon test). B. Relative abundance of Saccharomyces in the FMT and CTRL group at pre- and post-FMT. The paired mean difference between FMT pre—and post-FMT is −0.048 [95.0% CI −0.264, 0.167]; pValue>0.1 (Wilcoxon test). The paired mean difference between CTRL pre- and post-FMT is 0.0136 [95.0% CI−0.203, 0.28]; pValue>0.1 (Wilcoxon test). C. Non metric multidimensional scaling (NMDS) based on Bray-Curtis dissimilarities of the mycobiota community at different time points in the donor, Endoscopic non-responders (NR) and Endoscopic responders (Res). D. Pre-FMT Relative abundance of Candida among FMT recipient experiencing clinical response (Resp) and non-responders (NR). Two-sided P value, Mann-Whitney T test, * pValue=0.0185. E. Correlation between the pre-FMT relative abundance of Candida and the total Mayo Index post-FMT in placebo (CTRL) and FMT recipient (FMT). Lines represent linear regression for the placebo and FMT groups. Lower and upper hinges represent the 90% confidence bands. A,B,E. Cumming estimation plot of the paired mean difference, raw data is plotted on the upper panel; each set of paired observations is connected by a line. Lower panel: each paired mean difference is plotted as a bootstrap sampling distribution. Mean differences are depicted as dots; 95% confidence intervals are indicated by the ends of the vertical error bars. Effect size [CI width lower bound; upper bound].

FIG. 3. Changes in bacterial communities and association with fecal Candida levels. A. Simpson diversity index of 16s rRNA batches in patients at pre- and post-FMT. Patients were grouped based on the relative abundance of Candida (Ca) reads among fungal ITS reads pre-FMT. Low and high samples were defined as samples with a Candida relative abundance smaller and higher than the average Candida relative abundance (see also FIG. 4D). Two way ANOVA. Lower and upper hinges correspond to the 25th and 75th percentiles, dots represent individual samples. B. Upper panel: Relative abundance of bacterial phyla among 16s rRNA reads in FMT recipients pre-FMT. Lower panel: Relative Candida abundance in FMT recipients pre-FMT (showed as % of total ITS reads). C. Pearson correlation between bacterial genera in FMT recipients post-FMT and Candida abundance pre-FMT; grey band represent 95% confidence interval. Dots represent individual samples.

FIG. 4. A reduction in Candida abundance and associated immune-responses correlate with clinical and endoscopic response in FMT patients. A. Correlation between the change in relative abundance of Candida following FMT and the change in total Mayo Index following FMT in FMT recipient (FMT). Lines represent linear regression for the placebo and FMT groups. Lower and upper hinges represent the 90% confidence bands. B. Correlation between the change in relative abundance of Candida following FMT and the change in total Mayo Index following FMT in placebo (CTRL) patients. Lines represent linear regression for the placebo and FMT groups. Lower and upper hinges represent the 90% confidence bands. C. The paired mean difference for the anti C. albicans IgG in FMT and control patients before and after FMT shown as a Cumming estimation plot. The raw data is plotted on the upper axes; each paired set of observations is connected by a line. On the lower axes, each paired mean difference is plotted as a bootstrap sampling distribution. Mean differences are depicted as dots; 95% confidence intervals are indicated by the ends of the vertical error bars. The paired mean difference between pre- and post-FMT in FMT recipients is 0.0175 [95.0% CI−0.198, 0.181], pValue=0.141 (Wilcoxon test). The paired mean difference between pre- and post-FMT in Placebo recipients (CTRL) is 0.315 [95.0% CI 0.0802, 0.516], pValue=0.0186 (Wilcoxon test). D. Variation of anti C. albicans IgG in FMT and placebo (CTRL) recipients between pre- and post-treatment. The unpaired mean difference between FMT and Placebo is 0.41 [95.0% CI 0.109, 0.738], pValue=0.0215 (Mann-Whitney test).

FIG. 5. FMT enema ameliorates DSS induced colitis in C. albicans colonized mice. A. Mice were colonized with C. albicans for 2 consecutive days prior to the induction of DSS colitis. Daily enemas were performed from day 5 to day 9. % Weight loss from day 0. B. Colon length was determined at the end of the experiment. C. Representative pictures and quantification of the histological damage. Dots represent individual mice, line and bars represent mean+/−SEM.

FIG. 6. Mycobiota composition. A. Numbers of ITS reads recovered for the different patients' samples at the indicated time-points. B. Non metric multidimensional scaling (NMDS) based on Bray-Curtis dissimilarities of the mycobiota community at different time points in the donor (green), fecal transplantation recipient (FMT, red), and placebo recipient (CTRL, black) pre- and post-FMT. C. Alpha diversity measured by observed OTU for the different patients' samples at the indicated time-points. Relative abundance of Ascomycota (D), Basidiomycota (E), are shown for donor (green), fecal transplantation recipient (FMT, red), and placebo recipient (CTRL. black) pre- and post-FMT. (F) Relative abundance of C. albicans and non-C. albicans (other) Candida species across the analyzed samples. Dots represent individual patients, line and bars represent mean+/−SEM.

FIG. 7. Mycobiota composition in UC patients and healthy donors pre-FMT. A. Shannon diversity index, B. Log 10 (Ascomycota/Basidiomycota) Ratio. C. Relative abundance of Candida reads among total ITS fungal reads. D. Relative abundance of Saccharomyces reads among total ITS fungal reads. Data are shown as Gardner-Altman estimation plot of the mean differences between groups. Dot represent individual patients. Mean difference is plotted on floating axes on the right as a bootstrap sampling distribution. The mean difference is depicted as a dot; the 95% confidence interval is indicated by the ends of the vertical error bar.

FIG. 8. Relative abundance of major genera in the study cohort pre-FMT. Lower and upper hinges correspond to the 25th and 75th percentiles, dots represent individual samples. FMT: FMT recipients, CTRL: Placebo recipient. Shown are the Blinded Clinical response post-FMT: NR: non responders, Res: Responders.

FIG. 9. Candida and Saccharomyces abundance relative abundance. A. Relative abundance of Candida reads among total ITS fungal reads. The unpaired mean difference between FMT patients who met the primary endpoint at week 8 (RES) and patients who failed to meet the criteria (NR) is 0.19 [95.0% CI−0.0871, 0.456]. B. Relative abundance of Saccharomyces reads among total ITS fungal reads. The unpaired mean difference between NR and R is 0.106 [95.0% CI−0.176, 0.371]. Data in A, B, are shown as Gardner-Altman estimation plot of the mean differences between groups. Dot represent individual patients. Mean difference is plotted on a floating axes on the right as a bootstrap sampling distribution. The mean difference is depicted as a dot; the 95% confidence interval is indicated by the ends of the vertical error bar. C. Relative abundance of Candida in clinical non-responders (NR) and responders (R) of the FMT (red) and placebo (black) group pre—and post-FMT. The paired mean difference between pre- and post-FMT is 0.202 [95.0% CI −0.1, 0.51] in the FMT_NR group; −0.194 [95.0% CI−0.475, 0.131] in the FMT_R group; 0.0524 [95.0% CI−0.262, 0.33] in the Ctrl_NR group. pValue>0.1 (Wilcoxon test). Two-sided P value, Mann-Whitney T test is shown.

FIG. 10. Density distribution of Candida relative abundance in the analyzed patient samples. In FIG. 3. Samples with Candida relative abundance smaller than the geometric mean (<0.4347454) were defined as “low”. The remaining samples were defined as “high”.

FIG. 11. Ratio of clinical responders versus non-responders among FMT recipients post-FMT among patients with low Candida abundance (<10%) and high Candida abundance (>10%).

### DETAILED DESCRIPTION OF THE DISCLOSURE

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

This disclosure describes that fungal and bacterial counterparts of the microbiota are interdependent and that intestinal Candida colonization correlates with an increased diversity of the bacterial microbiota. Our data indicates that the presence of Candida supports enhanced bacterial engraftment during MTT. It is currently not possible to predict the clinical efficacy of MTT. Consequently, substantial effort is needed to identify factors that contribute to MTT treatment response that can be used as predictors prior to the initiation. The present findings indicate that the presence and levels of specific fungal species have predictive value as to the outcome of microbiome-based therapies, and can be used in increase the success of such therapies.

In more detail, the present methods and kits relate to use of MTT therapies, which involves using intestinal bacteria from a healthy or desired individual's fecal matter and then processing and transferring that bacteria to the afflicted patient directly. The fecal matter may be processed to extract/purify the bacteria. The transplantation of the fecal microbiota is generally carried out by colonoscopy, endoscopy, sigmoidoscopy, or enema. MTT may also be carried out by using frozen or freeze-dried fecal microbiota administered in pill form of any other ingestible form. The present disclosure provides a method of treating an individual afflicted with an irritable bowel disease. The method comprises (1) in a sample obtained from the individual's gut, determining the level of Candida sp. to be higher than a reference level; and administering to the individual, a therapy comprising microbiota transplantation therapy, or (2) in a sample obtained from the individual's gut, determining the level of Candida sp. to be lower than a reference level, administering a composition to the individual to increase the level of Candida sp. such that the Candida sp. levels increase to the same or higher than the reference level, and administering to the individual, a therapy comprising microbiota transplantation therapy.

The present disclosure provides a kit for treating an irritable bowel disease, the kit comprising (1) a microbiota transplantation therapy composition comprising fecal material or purified materials prepared therefrom obtained from one or more healthy individuals' gut; and (2) Candida sp. identification composition comprising one of more reagents to determine the level of Candida sp. to be higher or lower than a reference level.

The term “Microbiota transplantation therapy” or MTT as used herein means transfer of fecal material from one of more healthy donor or donors or a transfer of rationally designed combination of bacteria or metabolites from such fecal material. The term MTT encompasses FMT.

The term “isolated” or “purified” encompasses a bacterium or other entity or substance that has been (1) separated from at least some of the components with which it was associated when initially produced (whether in nature or in an experimental setting), and/or (2) produced, prepared, purified, and/or manufactured by the hand of man. Isolated bacteria may be separated from at least about 10%, about 20%, about 30%, about 40%, about 50%, about 60%, about 70%, about 80%, about 90%, or more of the other components with which they were initially associated. In some embodiments, isolated bacteria are more than about 80%, about 85%, about 90%, about 91%, about 92%, about 93%, about 94%, about 95%, about 96%, about 97%, about 98%, about 99%, or more than about 99% pure.

The term “Engraftment” of a type of bacterium, e.g., a species, is an effect of treatment with a composition of the disclosure that is characterized by post-treatment detection of a species from the administered composition, which is not detected in the treated subject pretreatment or is detected at a level higher than the pretreatment level. Methods of detection include PCR detection of a 16S rDNA sequence using standard parameters for PCR.

The terms “subject” or “patient” or “individual” refers to any animal subject including humans, laboratory animals (e.g., primates, rats, mice), livestock (e.g., cows, sheep, goats, pigs, turkeys, chickens), and household pets (e.g., dogs, cats, rodents, etc.).

As used herein the abbreviation “sp.” for species means at least one species (e.g., 1, 2, 3, 4, 5, or more species) of the indicated genus.

The term “therapeutically effective amount” as used herein refers to an amount of an agent sufficient to achieve, in a single or multiple doses, the intended purpose of treatment. Treatment does not have to lead to complete cure, although it may. Treatment can mean alleviation of one or more of the symptoms or markers of the indication. The exact amount desired or required will vary depending on the particular composition used, its mode of administration, patient specifics and the like. Appropriate effective amount can be determined by one of ordinary skill in the art informed by the instant disclosure using only routine experimentation. Within the meaning of the disclosure, “treatment” also includes relapse, or prophylaxis as well as the alleviation of acute or chronic signs, symptoms and/or malfunctions associated with the indication. Treatment can be orientated symptomatically, for example, to suppress symptoms. It can be effected over a short period, over a medium term, or can be a long-term treatment, such as, for example within the context of a maintenance therapy. Administrations may be intermittent, periodic, or continuous.

The term “pharmaceutically acceptable” is employed herein to refer to those compounds, materials, compositions, and/or dosage forms which are suitable for use in contact with the tissues of the subject with toxicity, irritation, allergic response, or other problems or complications, commensurate with a reasonable benefit/risk ratio.

In an embodiment, the disclosure provides a method of identifying if an individual if suited for MTT. The method comprises determining the level of Candida sp. in a sample obtained from the GI tract of an individual. The sample may be collected from a specimen of excrement or by an invasive procedure (e.g., introduction of devices (such as during colonoscopy), into the GI tract by which a sample of the tissue or materials being processed by the GI tract can be collected).

A sample from the gastrointestinal tract can be collected by any routine means, such as fecal sample, anal swab, intestinal biopsy, fecal washing or mucosal washing and the like. The sample can be used for determining Candida sp. levels.

The presence and level of Candida may be determined by any routine method known in the art for detecting Candida in biological samples. For example, Candida may be detected at the antigen level by using specific antibodies which may be directly or indirectly detectably labeled, or by the use of anti-fungal drugs or any other specific fungal binding agents which may be detectably labeled. Candida may also be detected by using carbohydrate assimilation and fermentation reactions, or by using culture based techniques. Candida may also be detecting and quantified by using DNA PCR, RT-PCR. In an embodiment, the presence and level of Candida sp. is determined by RT-qPCR, where fungal ITS1-2 regions may be amplified, or multiKAP assay.

In an embodiment, level of Candida sp. is determined based on 16S rRNA, whole genome sequence, any subset of a genome as well as whole genome shotgun metagenomic sequence (WGS). Sequence data may be generated using any sequencing technology including, but not limited to Sanger, Illumina, 454 Life Sciences, Ion Torrent, ABI, Pacific Bioscienses, and/or Oxford Nanopore.

In an embodiment, level of Candida sp. is determined by Multi-Kingdom Antibody Profiling (multiKAP) assay, wherein fungal and/or bacterial content can be determined by using antibodies. For example, fecal samples may be collected and diluted in a suitable buffer(e.g., PBS), homogenized, and filtered (e.g., through a 35 m strainer-capped tube). Resulting fecal slurries can be incubated for a period of time (e.g., for 90 minutes at 30° C. with serum) and diluted at certain dilutions (e.g., 1:100 (human serum)) in blocking buffer (1% bovine serum albumin (BSA)/0.05% sodium azide). After a period of time (e.g., 30-minutes of blocking with staining buffer (2% BSA/0.05% sodium azide)), samples can be stained with antibodies (e.g., 1:200 hIgA-PE (Miltenyi) and/or 1:400 hIgG-AF647 (Southern) antibodies). Total fungal and bacterial content is analyzed by flow cytometry. Size separation can be achieved by centrifugation at 900×g for 10 minutes. The resulting large (fungal) and small (bacterial) IgA- and/or IgG-stained fractions can be used for flow cytometry analysis and/or sorting and/or DNA isolation. In a variation of the method, size separation may be performed as a first step followed by serum incubation and antibody staining as above. For IgG isotype multiKAP assays, samples can be stained with mIgG1-FITC (e.g., from Thermofisher), mIgG2b-FITC (e.g., from Thermofisher), or mIgG3-FITC (e.g., from BD Pharmingen). To avoid spectral overlap between FITC and Sybr-Green dye when FITC-labeled antibodies are used, DNA staining can be performed with propidium iodide (PI; e.g., from Thermofisher) or DAPI as an alternative DNA-binding dyes. Antibody bound (IgG+IgA+, IgG+IgA−, IgG−IgA+) and unbound (IgG−IgA−) fungal and bacterial fractions can be sorted on FACSAria III (e.g., from BD Bioscience) with a flow cell and processed for DNA isolation and microbiota (fungal and bacterial) qPCR and sequencing. The detection of Candida may be a broad based detection of a plurality of Candida species, or one or more representative species found in the GI tract, e.g., C. albicans.

The detected Candida levels can be compared to reference levels, which may be levels from healthy individuals who do not have IBD.

In an embodiment, the level of Candida sp. is determined by ITS1 fungal reads, 16S bacterial analysis, multiKAP assay, or a combination thereof. In an embodiment, the reference level is determined by a mean relative abundance of Candida sp. level from a control group of individuals.

In an embodiment, the disclosure provides a method of treating an individual afflicted with IBD. The IBD may be ulcerative colitis or Crohn's disease. The method comprises determining level of Candida in a sample from the GI tract of an individual. In an embodiment, the sample from the individual's GI tract is obtained from a stool or by biopsy.

Following determination of Candida levels in GI tract (e.g., stool samples) from an individual, the individual may be identified as being suited for MTT. The MTT therapy may comprise administration of compositions comprising intestinal microbiota from one or more healthy individuals. The intestinal microbiota may be in the form of fecal matter, or fractions thereof. The fecal matter may be processed, and transplanted into the GI tract of afflicted individual. For example, the transplantation of the fecal microbiota is generally carried out by colonoscopy, endoscopy, sigmoidoscopy, enema, or administered in pill or other ingestible form by using fecal materials or materials comprising microbiota prepared therefrom, including frozen or freeze-dried fecal microbiota. Additionally, the fecal matter may include metabolites of the GI tract microbiome, bacteria, viruses, fungi, archea, GI tract microbiome metabolites, or a mixture thereof. MTT may comprise either transfer of fecal material form one or more healthy donor(s) or a transfer of isolated bacteria and/or other microbes or rationally designed combination of bacteria, or metabolites.

In an embodiment, suitable microbiota for engraftment for the present disclosure may be isolated from fecal material. Methods of isolation and purification of microbiota from fecal material are known in the art and may include one or more of centrifugation, filtration, fractionation, affinity based isolation and the like. The process of preparing may include collecting a fecal sample from one or more donors, processing the fecal sample(s) to isolate and purify the selected bacteria, optionally lyophilizing or “freeze-drying” the processed fecal sample (or otherwise converting the processed fecal sample from a liquid to a solid or powdered, or dehydrated form), adding one or more additives and/or excipients, and forming a desired form (such as oral form or form deliverable via enema) of the composition. The isolated, purified bacteria may be in a lyophilized or freeze dried form by itself or the composition may comprise materials and additives suitable for oral administration (e.g., a tablet, capsule, liquid preparation, or the like) or otherwise delivery to the GI tract. Other forms for administration via other routes (e.g., rectal) may alternatively or additionally be used.

In an embodiment, pharmaceutical composition can be formulated for oral administration of MTT. The present oral compositions may be in the form of a chewable formulation, a dissolving or dissolved formulation, an encapsulated/coated formulation, a multi-layered lozenges (to separate active ingredients and/or active ingredients and excipients), a slow release/timed release formulation, or other forms suitable for oral delivery known in the art. It may be in the form of a tablet, lozenges, pill, capsule, drops, liquid form, enema or the like. The formulations may also be present as encapsulated or incorporated into micelles, liposomes, cyclodextrins, polymers and the like. The pharmaceutical compositions, including pediatric formulations, may be flavored (e.g. fruit flavored, such as cherry, strawberry, blueberry, etc.) and may be in a variety of shapes or colors. Excipients may be added to the formulations. For example, binders include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch. Sweetening agents such as sorbitol, saccharin, sodium saccharin, sucrose, aspartame, fructose, mannitol and invert sugar may be added to improve the taste. Flavoring agents and flavor enhancers may make the dosage form more palatable to the patient. Common flavoring agents and flavor enhancers for pharmaceutical products that may be included in the composition of the present disclosure include maltol, vanillin, ethyl vanillin, menthol, citric acid, fumaric acid, ethyl maltol and tartaric acid. Preservatives and chelating agents such as alcohol, sodium benzoate, butylated hydroxy toluene, butylated hydroxyanisole and ethylenediamine tetraacetic acid may be added at levels safe for ingestion to improve storage and stability.

MTT may be carried out as needed. For example, it may comprise 1 or more doses per week for several weeks. It may comprise 1 dose to 5 or more doses a week, which can be continued for at least 2 weeks, such as up to 8 or 10 weeks, or more.

In individuals, who have low levels of Candida such that they are not identified to be suitable directly for MTT, a composition (such as a probiotic) comprising one or more Candida sp. can be administered or delivered to the GI tract to increase the GI tract Candida levels. Upon increase of GI tract Candida levels, the individual can be subjected to MTT therapy. The compositions may comprise an effective amount of one or more of the Candida species so as to increase the level of Candida sp. in the GI tract. The Candida species may be formulated together with one or more pharmaceutically acceptable excipients and may be formulated into compositions and dosage forms according to methods known in the art. The pharmaceutical compositions comprising Candida may be specially formulated for administration in solid or liquid form, including those adapted for the following: oral administration, for example, tablets, capsules, powders, granules, and aqueous or non-aqueous solutions or suspensions, drenches, or syrups, frozen or freeze-dried forms; or intrarectally, for example, as a pessary, cream or foam. The compositions, which may be probiotic compositions, may be present in a lyophilized form (i.e., freeze-dried form). The Candida sp. may be lyophilized individually or the entire probiotic composition may be lyophilized.

In an embodiment, the present disclosure provides a method of identifying if an individual afflicted with IBD is suited for a GI tract microbiota-derived treatment (e.g., MTT) comprising: determining the level of Candida in a sample obtained from the individual's GI tract comprising microbiota, if the level is the same or higher than a reference level, then identifying the individual to be suitable for the GI tract microbiota-derived treatment, such as microbiota transplantation therapy; or if the level is lower than the reference level, then identifying the individual to be not suitable for the GI tract microbiota-derived treatment, and optionally, administering a composition to the individual to increase the GI tract Candida levels, and then administering the MTT. After administering a composition to increase the GI tract Candida levels, a determination of the Candida levels in the GI tract may be carried out to confirm that an increase has occurred prior to MTT. In an embodiment, the presence or levels of a plurality of Candida sp. may be tested. In an embodiment, the presence or levels of one or more specific species, such as C. albicans, may be tested.

In embodiments, Candida levels in samples from the GI tract may be analyzed on an ongoing manner to monitor the levels for MTT suitability, Candida level evaluations, or checking efficacy of MTT purposes.

In an embodiment, the present disclosure provides a method of treating an individual afflicted with IBD comprising: (a) in a sample obtained from the individual's GI tract, determining the level of Candida to be the same or higher than a reference level; and (b) administering to the individual, a therapy comprising fecal matter transfer or MTT, wherein the fecal matter is obtained from one or more healthy individuals. In an embodiment, instead of fecal matter, microbiota and/or one or more metabolites isolated from the fecal matter may be administered.

In an embodiment, the present disclosure provides a method for treating an individual afflicted with IBD comprising: (a) in a sample obtained from the individual's GI tract, determining the level of Candida to be lower than a reference level; (b) administering a composition to the individual to increase the level of GI tract Candida such that the Candida levels in the GI tract increase to the same or higher than the reference level; and (c) administering to the individual, a therapy comprising fecal matter transfer or MTT, wherein the fecal matter is obtained from one or more healthy individuals. In an embodiment, instead of fecal matter, microbiota isolated from the fecal matter may be administered.

In an embodiment, the present disclosure provides a method of treating IBD, the method comprising: obtaining a sample of the individual's GI tract microbiota; determining the level of Candida in the GI tract microbiota; (1) if the level of Candida is the same or higher than a reference level, administering the individual a GI tract microbiota-derived treatment; optionally, monitoring the individual's response to the treatment until the GI tract microbiota induced condition is ameliorated; or (2) if the level of Candida is lower than the reference level, giving the individual an agent to raise the level of Candida; and then administering to the individual a GI tract microbiota-derived treatment; optionally, monitoring the individual's response to the treatment until the gut microbiota induced condition is ameliorated. The agent to raise the level of Candida may be medical, pharmaceutical, or probiotic composition.

In an aspect, the present disclosure provides kits for treating an intestinal bowel disease. In an embodiment, the kit comprises (a) one or more reagents for determining the presence and levels of Candida sp. in a biological sample; and (b) a fecal microbiota transplantation therapy composition. Optionally, instructions may be provided for use of the components, and a reference standard level for comparison of Candida levels. The reagents for determining presence and levels of Candida may be agents for binding based detection (e.g., antibodies or anti-Candida drugs or agents which may be directly or indirectly detectably labeled), or materials for nucleic acid based detection (e.g., primers and reagents for specifically detecting Candida via RT-PCT, qPCR and the like). The MTT composition may be ready to use composition in any suitable form, including, but not limited to, capsules, tablets, pills, powdered, liquid or any other form. Optionally, the kit may further comprise a composition for increasing GI tract Candida levels, which may be in the form of a probiotic, an ingestible pill, tablet, capsule, liquid, powder and the like. The kit may optionally contain instructions for use of the ingestible compositions. The kit may optionally provide information on reference levels for comparison of GI tract Candida levels.

The individual or subject that may be administered a composition of the present disclosure may be any animal, including human and non-human mammals. The subjects, such as human subjects may suffering from IBD or at risk for IBD. The human may be of any age or gender. The subject is generally diagnosed with an inflammatory bowel disease by skilled artisans, such as a medical practitioner.

A therapeutically effective amount of the pharmaceutical composition of the present disclosure is sufficient to promote the health of the gastrointestinal tract, or to alleviate one or more symptoms of IBD. The dosage of active ingredient(s) may vary, depending on the individual subject. The dosage may be adjusted based on the subject's weight, the age and health of the subject and this is well within the purview of those skilled in the art.

The appropriate dosage and treatment regimen of the probiotic or other Candida compositions or MTT may be determined or recommended by a clinician or nutritionist. In general, one or more doses may be administered per day for a day, week, and month or longer if needed. For example, a dose may be administered every day for 1 week. In embodiments the compositions may be provided via enema or oral route. The treatment may be carried out as long as needed for the symptoms to alleviate or may be used as a maintenance therapy.

The following examples are provided as illustrations of the present disclosure and not intended to be limiting.

### Example 1

Previously, in a large multi-center, double-blind, randomized, placebo-controlled FMT trial in UC, steroid-free clinical remission was achieved in 44% (18 of 41) of patients receiving multidonor intensive heterologous MTT versus 20% (8 of 40) of those receiving placebo (Paramsothy et al. 2017, The Lancet 389 (10075):1218-1228). However, as in other trials for FMT in UC, no specific factors were identified that influenced the observed bacterial engraftment and treatment response.

Methods

DNA isolation, fungal rDNA amplification, Illumina library generation and sequencing. Fecal samples from donor and recipients were collected. Samples from 24 FMT recipients and 19 placebo recipients were processed. DNA for fungal sequencing was isolated from 50-80 mg material following lyticase treatment, bead beating, and processing using the Quick-DNA Fungal/Bacterial Kit (Zymo Research). Presence or absence of fungal DNA was validated in each sample by RT-qPCR. Fungal ITS1-2 regions were amplified using modified primers that include sample barcodes and sequencing adaptors as shown below in Table 1.

TABLE 1
Primer Sequences

| Primer | Sequence |
|---|---|
| ITSIF | CTTGGTCATTTAGAGGAAGTAA |
| | (SEQ ID NO: 1) |
| ITS2R | GCTGCGTTCTTCATCGATGC |
| | (SEQ ID NO: 2) |
| Forward | 5′-TCGTCGGCAGCGTCA |
| overhang | GATGTGTATAAGAGACAG |
| | (SEQ ID NO: 3)- |
| | [locus-specific sequence] |
| Reverse | 5′-GTCTCGTGGGCTCGG |
| overhang | AGATGTGTATAAGAGACAG |
| | (SEQ ID NO: 4)- |
| | [locus-specific sequence] |

Specifically, ITS amplicons were generated with 35 cycles using Invitrogen AccuPrime PCR reagents (Carlsbad, Calif.). Amplicons were then used in the second PCR reaction, using Illumina Nextera XT v2 (San Diego, Calif.) barcoded primers to uniquely index each sample and 2×300 paired end sequencing was performed on the Illumina MiSeq (Illumina, CA). DNA was amplified using the following PCR protocol: Initial denaturation at 94° C. for 10 min, followed by 40 cycles of denaturation at 94° C. for 30 s, annealing at 55° C. for 30 s, and elongation at 72° C. for 2 min, followed by an elongation step at 72° C. for 30 min.

All libraries were subjected to quality control using qPCR, DNA 1000 Bioanalyzer (Agilent), and Qubit (Life Technologies) to validate and quantitate library construction prior to preparing a Paired End flow cell. Samples were randomly divided among flow cells to minimize sequencing bias. Clonal bridge amplification (Illumina) was performed using a cBot (Illumina) and 2×250 bp sequencing-by-synthesis was performed.

ITS1 fungal reads and 16S bacterial analysis. The generated raw FASTQ ITS1 sequencing data were filtered to enrich for high quality reads, removing the adapter sequence by cutadapt v1.4.1 or any reads that did not contain the proximal primer sequence. Sequence reads were then quality-trimmed by truncating reads not having an average quality score of 20 over a 3 base pair sliding window and removing reads shorter than 100 bp. These reads were then aligned to Targeted Host Fungi (THF) ITS1 database, using BLAST v2.2.22 and the pick_otus.py pipeline in the QIIME v1.6 wrapper with an identity percentage>97% for OTU picking. The alignment results were subsequently tabulated across all reads, using the accession identifier of the ITS reference sequences as surrogate OTUs and using a Perl script. Among the analyzed samples, 23 were excluded due to insufficient quality size (ITS reads<200). Bacterial reads were analyzed. The R packages Phyloseq (1.26.1) and Vegan (2.5-5) were used for determining community properties such as Shannon index, Simpson index, Bray-Curtis index, NMDS scaling of Bray-Curtis dissimilarities, and relative abundance at various taxonomic levels. R version 3.5.0 was used.

Detection of anti-Candida antibodies in the human serum. The level of antibodies in the human serum that react with Candida antigens was determined by a sandwich enzyme-linked immunosorbent assay (ELISA). Briefly, to obtain bacterial or fungal lysates for ELISA the overnight Candida albicans SC5314 (ATCC MYA-2876) colonies were prepared through freeze and thawing disruption followed by 5 pulses of sonication. Candida lysates were used as the coating antigen in ELISA assay on high binding flat bottom plates (EIA/RIA 96-Well Plates, Corning). ELISA detection of IgG was carried out using Alk-Phosphatase coupled goat anti-Mouse IgG (Jackson Lab) and PNPP substrate (Life technologies). Samples were read at 405 nm on a microtiter plate reader (Menlo Park, Calif.).

Experimental replication, randomization and blinding. Age matched groups of mice were randomly allocated to the experimental groups. To ensure reproducibility, the mouse fecal transplant experiment was performed independently twice.

DSS-induced intestinal inflammation. Age-matched female C57BL/6J mice of 10 weeks of age were purchased from the Jackson Laboratories (BarHarbor, ME) and subsequently housed for 2 weeks under specific pathogen-free (SPF) conditions in a negative pressure con-trolled environment room at Weill Cornell Medicine (WCMC). All experiments were conducted with at least four mice per group with prior approval from the Institutional Care and Use Committee of WCMC. Mice were orally supplemented with C. albicans (5*10∂yeast in 200 ul) for 2 consecutive days prior to the DSS treatment. Control groups were orally gavaged with PBS at the same time points. Body weight and the presence of occult blood were assessed daily. Mice were administered with sterile 3% dextran sodium sulphate (DSS, Sigma) for 7 days followed by a recovery period of 3 days with water. Starting at day 4 post DSS initiation, daily enema where administered daily to the experimental mice. For enema, mice were anesthetized with isoflurane and intrarectally administered freshly prepared 200ul of a microbial suspension prepared from fresh pellets collected from 3 different sex and age-matched C57B16/J donor mice (90 mg in 1 ml sterile PBS) filtered through a 40 um sterile nylon mesh. Control mice were anesthetized with isoflurane and intrarectally administered 200ul of sterile PBS.

Mouse dissection and preparation of cells. Mice were sacrificed by C02 followed by cervical dislocation. Mesenteric lymph nodes (mLN) and large intestine were dissected and placed in cold PBS until further processing. Colon length was measured and a distal 0.4 cm segment was fixed in 4% paraformaldehyde for histological analysis. mLN were strained through a 40 um filter cap, centrifuged at 580 rcf for 5 min, resuspended in PBS, counted and stained as described below. For intestinal LP cells isolations, colons were opened longitudinally, washed of fecal contents and then cut into 1 cm pieces. Intestinal pieces were transferred into HBSS medium (Sigma), supplemented with 5% fetal bovine serum (FBS) and 2 mM EDTA, and were shaken for 8 min at 37° C. The remaining tissue was washed, cut in small pieces and subsequently incubated in digestion medium consisting of RPMI 1640, 5% FBS, 0.5 mg/ml collagenase type VIII (Sigma), 5 U/ml DNase (Roche Diagnostics), 100 IU/ml penicillin and 100 μg/ml streptomycin for 25 min at 37° C. by gentle shaking. The cell suspensions were filtered through a 70 um mesh, centrifuged at 580 rcf for 7 min at 20° C., washed twice with PBS, and used for immunophenotyping.

Antibodies and flow cytometry. Cell suspensions were prepared as described above, blocked with CD16/CD32 (Mouse BD Fc Block™, 2.4G2, BD Biosciences) and stained with antibodies against CD4 (GK1.5, eBioscience), CD45 (30-F11, Tonbo), CD90.1 (OX-7, Biolegend), CD11b (M1/70, Tonbo), CD11c (N418, Tonbo), CD64 (X54-5/7.1 FC, Biolegend), CD103 (2E7, eBioscience), MHC-II (I-A/I-E, M5/114.15.2, eBioscience), NK1.1 (eBioscience, PK136), Ly-6G (1A8, Biolegend), CX3CR1 (SA01 IF 11, Biolegend), CD40 (HM40, eBioscience), CD86 (Gl-1, Biolegend), Dectin-1 (2A11, Bio-Rad), Dectin-2 (KVa7-6E7, Miltenyi). For intracellular staining of transcription factors, cells were stained with surface markers, fixed permeabilized and stained with FoxP3 (FJK-16s, eBioscience) and RORgt (B2D, eBioscience). For intracellular cytokine and Syk detection, cells were incubated with 50 ng/ml phorbol 12-myristate 13-acetate (PMA; Sigma-Aldrich), 500 ng/ml ionomycin (Sigma-Aldrich) and 10 μg/ml Brefeldin A (BFA; Sigma-Aldrich) in complete RPMI media at 370° C. for 6 h. After surface staining with CD45, CD4, Thy1.1 cells were fixed, permeabilized and staining was performed using PE-labeled anti-IL-17 mAb (eBiol7B7; eBiosciences) or PE-labelled anti-Syk mAb (5F5; Biolegend) according to the manufacturer's instructions. Flow cytometry was performed using a LSRFortessa (BD Biosciences) and data were analyzed with FlowJo software (TreeStar Inc.).

Quantification and Statistical Analysis. Unless otherwise indicated, statistics were computed using the dabestR package for Estimation Statistic in R. 5000 bootstrap samples were taken; the confidence interval is bias-corrected and accelerated. Statistical details of experiments are reported in the figure legends. The P value(s) reported in the figure legends are the likelihood(s) of observing the effect size(s), if the null hypothesis of zero difference is true.

Data and Code Availability. The published article includes all datasets generated and analyzed during this study. Fecal ITS rDNA amplicon sequencing data were submitted to the Sequence Read Archive to the NCBI BioProject repository (BioProject ID PRJNA590898). Fecal 16S rDNA amplicon sequencing data were submitted as part of Paramsothy et al, 2017 to to the European Nucleotide Archive (accession numbers PRJEB26472, PRJEB26474, PRJEB26473, PRJEB20349 and PRJEB26357).

### EXPERIMENTAL MODEL AND SUBJECT DETAILS

Human Subjects. Patient samples were obtained from a previously reported double-blind, controlled trial of FMT in UC patients. Briefly, male and female patients aged 18-75 years were subjected to an initial colonoscopic infusion and then intensive multidonor FMT (32 patients) or placebo enemas (29 patients) 5 days/week for 8 weeks. Fourteen donors were used for the study, with random selection of 3-7 fixed donors for each of the 21 multidonor FMT batches.

Patients had to have clinically and endoscopically active UC, with a total Mayo score of 4-10. The Mayo endoscopy subscore had to be 1 or greater and physician's global assessment subscore 2 or less. We included any disease extent except proctitis confined to the distal 5 cm. We excluded individuals with indeterminate colitis, major comorbid chronic disease, major food allergy, irritable bowel syndrome, or a history of bowel cancer, those who were pregnant, and patients who had previous gastrointestinal surgery apart from appendectomy more than 3 months before the study. We excluded gastrointestinal infection at study entry, including parasitic and C. difficile infections.

We permitted the following drugs as long as the dose was stable preceding enrolment: oral 5-aminosalicylates (stable dose for 4 weeks); thiopurines and methotrexate (on medication for >90 days and dose stable for 4 weeks); and oral prednisone (dose<20 mg daily and stable for 2 weeks). During the study, patients remained on the same dose of 5-aminosalicylate, thiopurine, and methotrexate. For oral prednisone, we did a mandatory taper of up to 2.5 mg per week so that patients would be steroid-free by week 8. We did not allow rectal therapies, including: corticosteroids or 5-aminosalicylate (for 2 weeks before enrolment); antibiotics or probiotics (for 4 weeks before enrolment); and biological therapies or calcineurin inhibitors (for 12 weeks before enrolment).

FMT infusions were constituted from the blended homogenized stool of 3-7 unrelated donors to increase microbial heterogeneity. Each patient received all of their FMT infusions from the same donor batch comprising fixed individual donors. This investigator-initiated study was sponsored by the University of New South Wales, approved by the St Vincent's Hospital Sydney Human Research Ethics Committee (HREC/13/SVH/69), and registered with ClinicalTrials.gov (NCT01896635) and the Australian Therapeutic Goods Administration Clinical Trial Notification Scheme (2013/0523).

Results

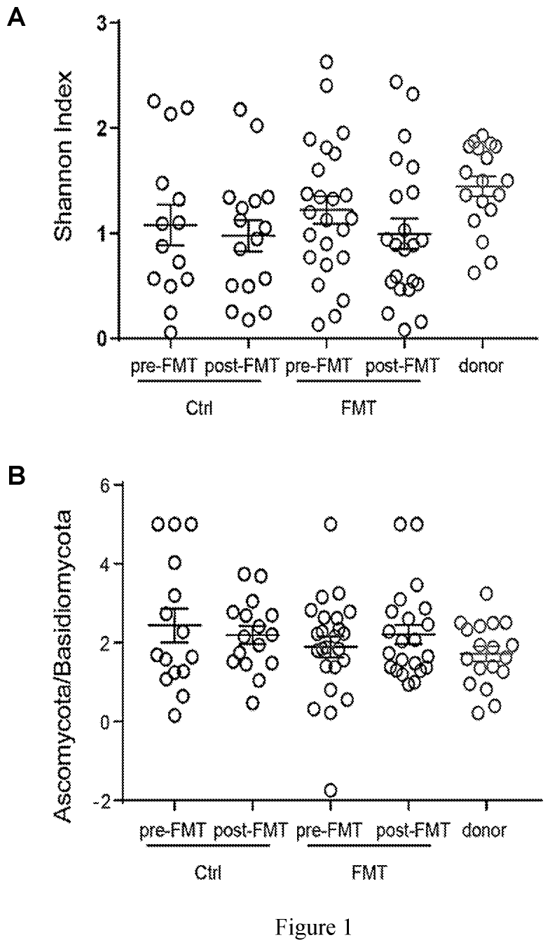

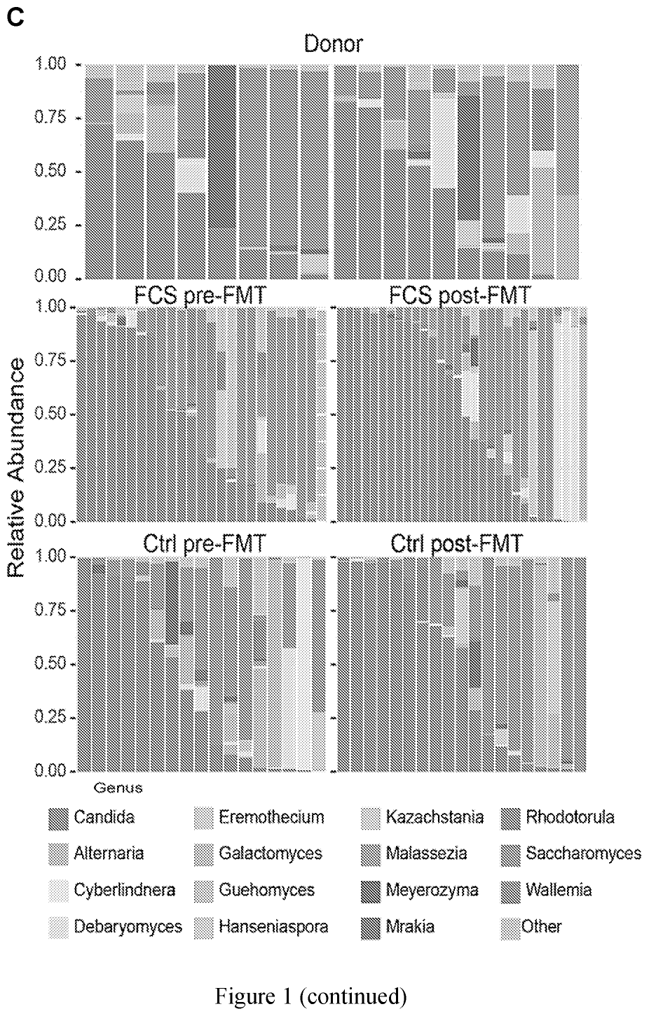

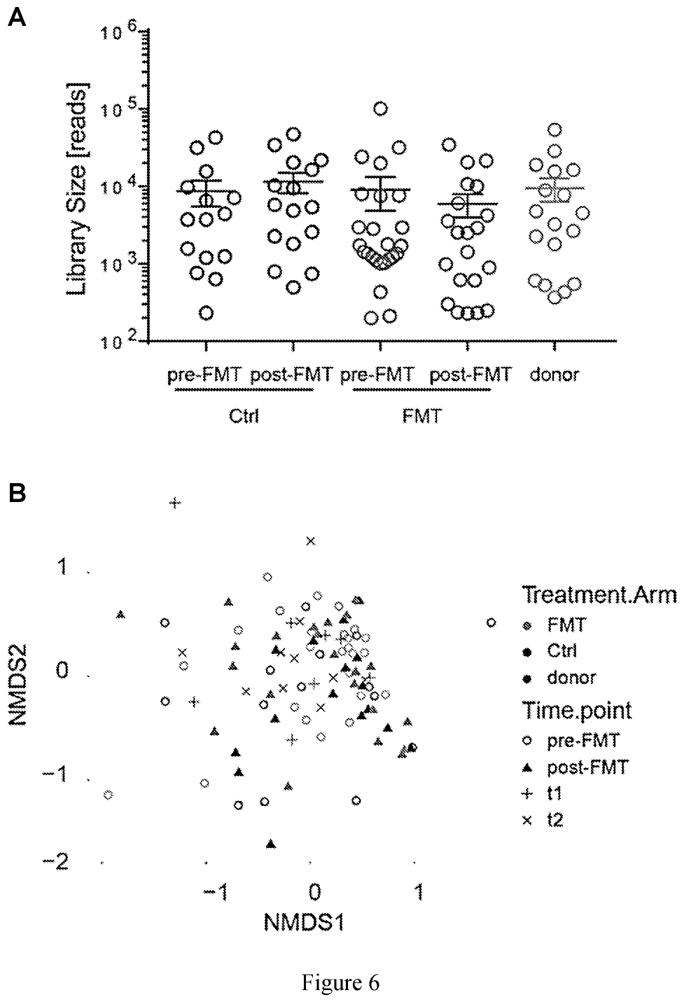

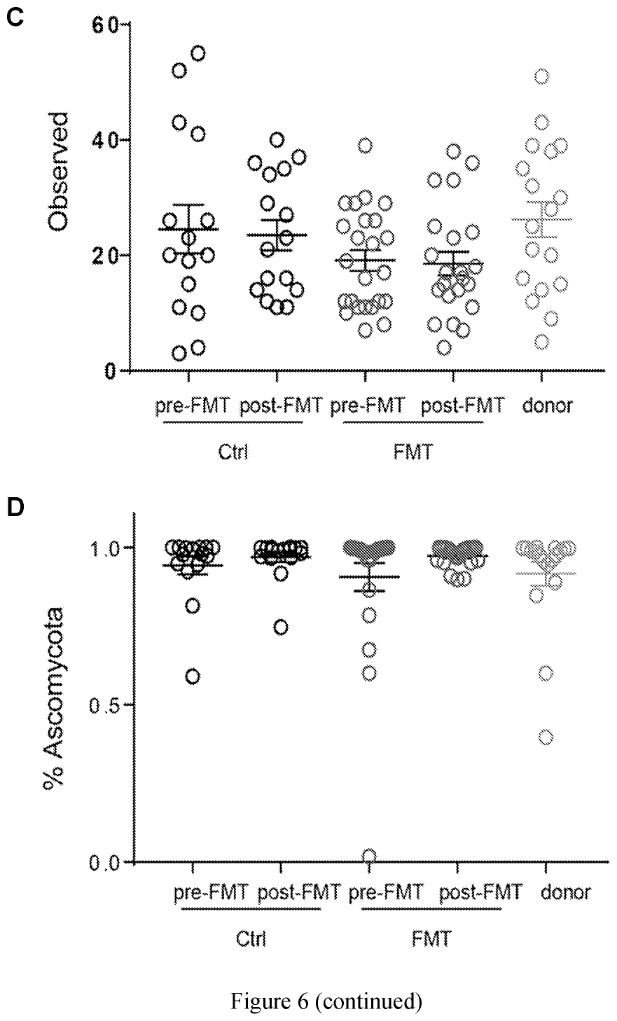

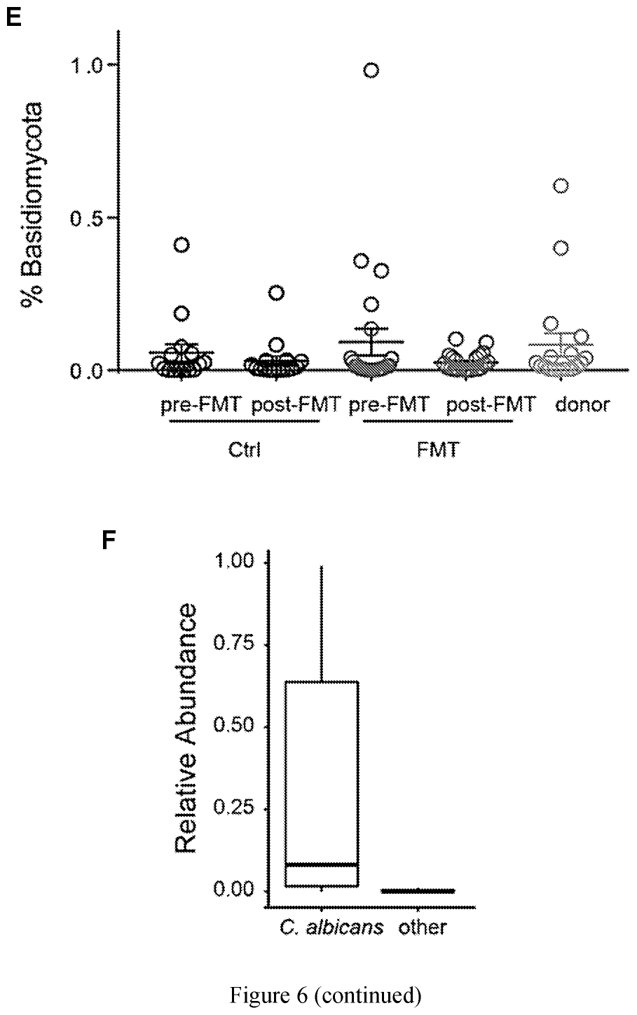

To investigate the dynamics of the intestinal mycobiota in UC patients undergoing FMT we used an Internal Transcribed Spacer (ITS)-1 based barcoding approach to deep sequence the ITS-1 regions of fungal rDNA in fecal samples from the FOCUS study. Specifically, ITS sequencing was performed on 129 fecal samples collected from FMT donors and UC patients pre-FMT and following 8 weeks of intensive FMT or placebo treatment (post-FMT). ITS sequencing resulted in a uniform library size distribution across the analyzed samples (FIG. 6A, Table 1). The mycobiota was generally dominated by the Ascomycota phylum in all groups (FIG. 6D-E). We observed a trend towards lower fungal alpha-diversity and increased Ascomycota/Basidiomycota ratio in feces from UC patients pre-FMT when compared to healthy donors (FIG. 1A, B and FIG. 6C, 7A, B). However, these differences did not reach statistical significance, possibly due the mild-moderate nature of UC in our patients' cohort.

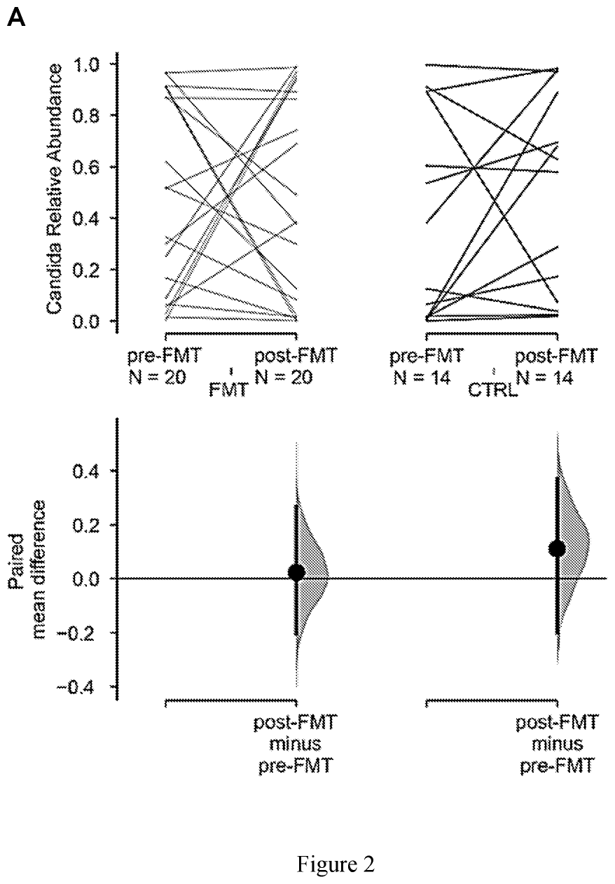

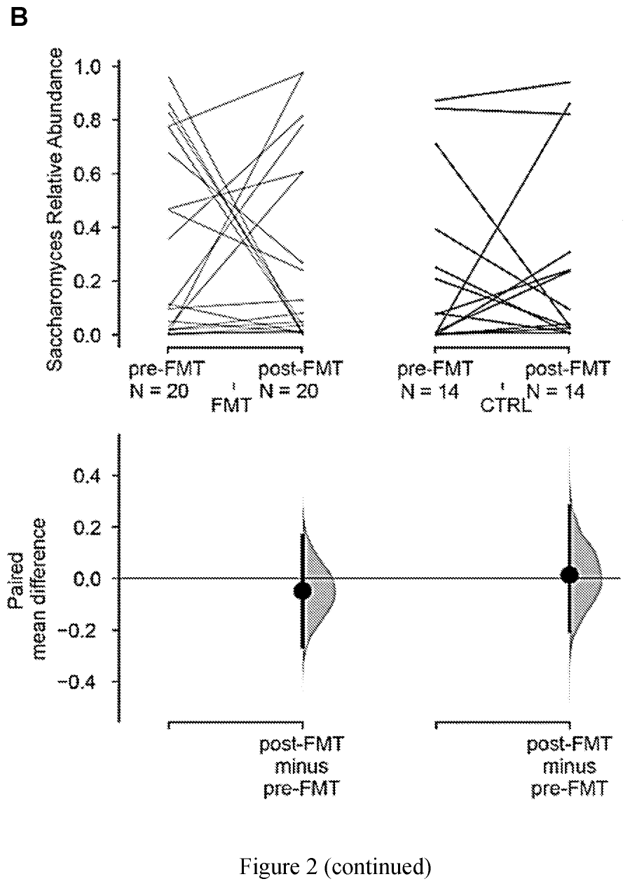

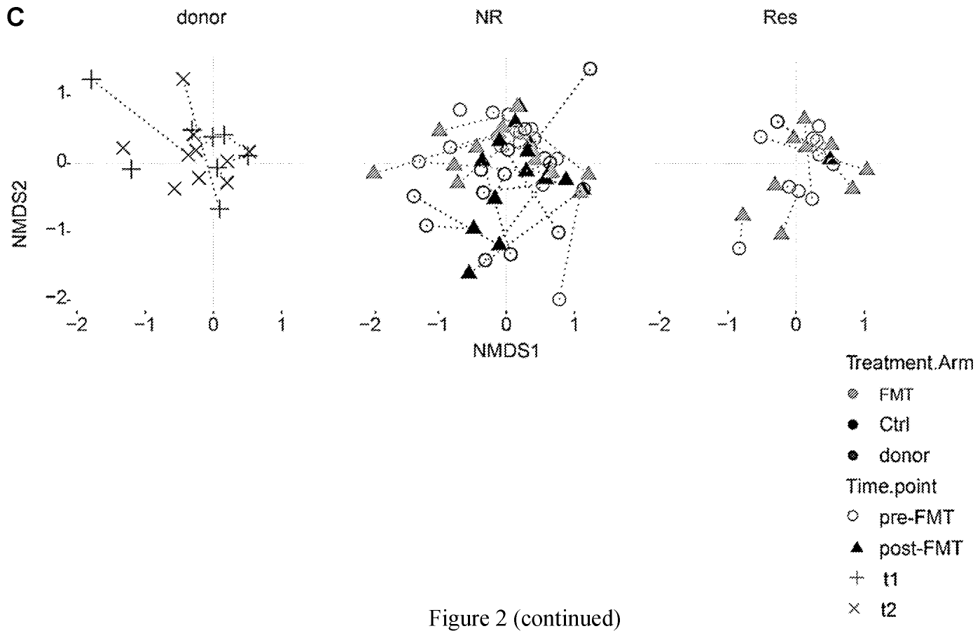

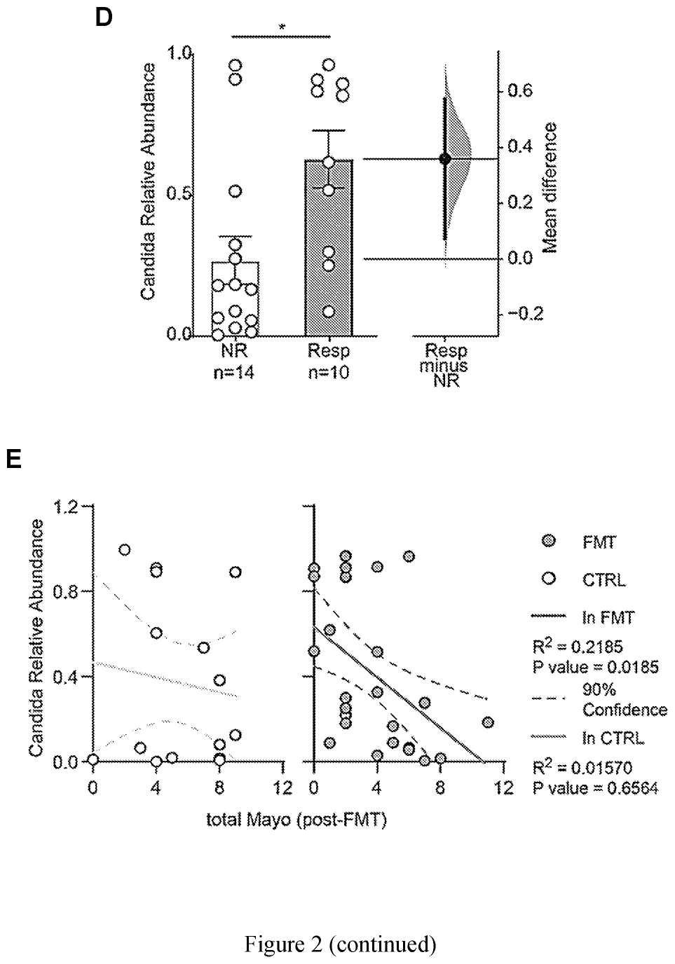

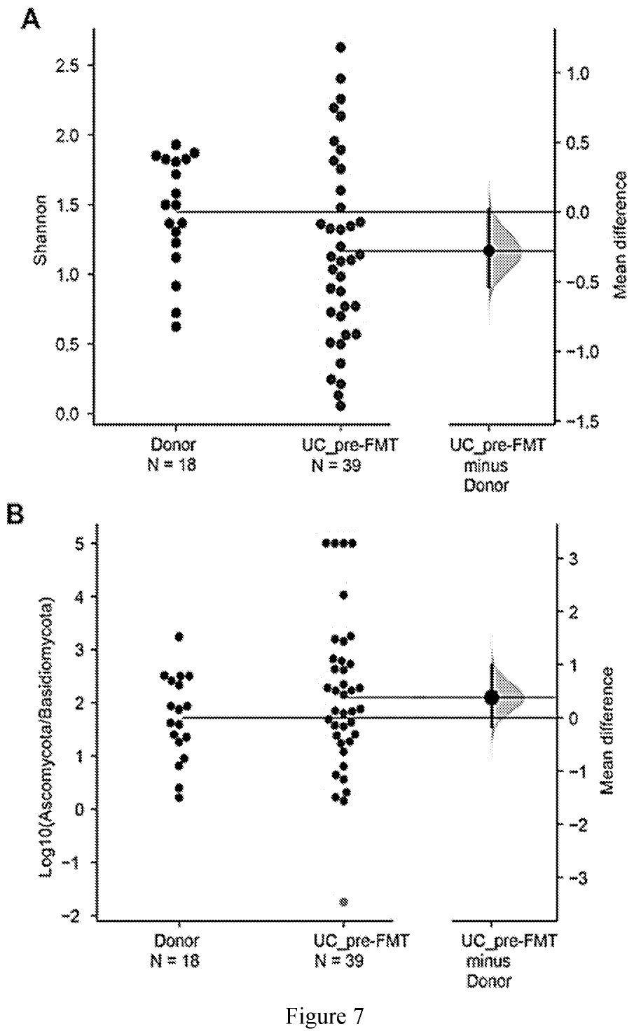

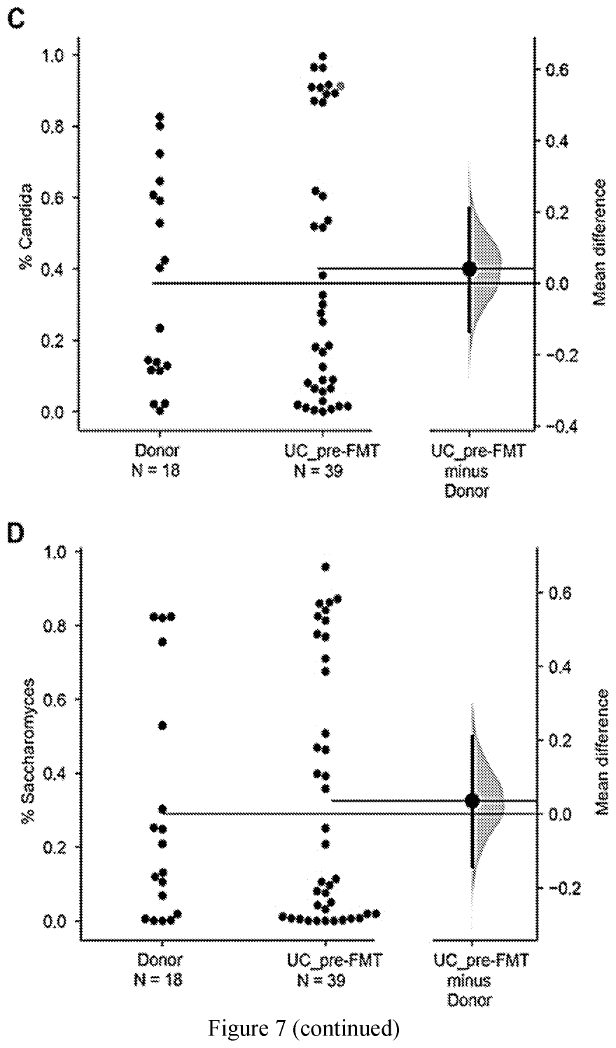

Although the bacterial composition changed post-FMT by both increased observed OTUs and diversity relative to baseline, the gut mycobiota diversity, the abundance of the two major fungal phyla and observed OTUs all remained unaltered, suggesting that FMT did not affect the overall structure of gut fungal communities in our UC cohort. A deeper analysis of the mycobiota taxonomic composition revealed 15 major fungal genera with Candida and Saccharomyces being the most abundant in both healthy donors and UC patients (FIG. 1C, FIG. 7C, D). Within the Candida genus, Candida albicans was the most abundant and prevalent species (FIG. 6F). Both Candida and Saccharomyces showed a similar relative abundance between donors and recipients pre-FMT (FIG. 7C, D). We observed that in contrast to previously reported post-FMT mycobiota outcomes in a CDI cohort (Zuo et al. 2018, Nature Communications 9 (1):1-11. doi: 10.1038/s41467-018-06103-6), multi-donor intensive FMT did not affect the relative abundance of Candida and Saccharomyces post-FMT in the UC recipients (FIG. 2A, B).

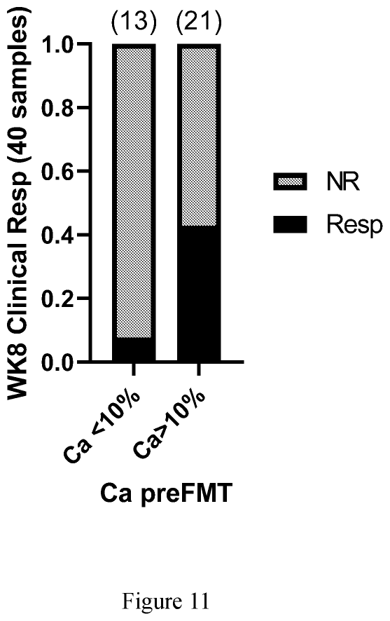

To investigate if pre-existing intestinal landscape can dramatically affect the outcome of FMT, we next assessed the composition of the gut mycobiota pre-FMT and analyzed correlations with the clinical outcomes post-FMT therapy. While FMT responders and non-responders did not cluster separately based on overall taxonomic composition (FIG. 2C), clinical FMT response was associated with significantly higher levels of Candida in pre-FMT samples compared to samples from FMT non-responder recipients (FIG. 2D, FIG. 11).

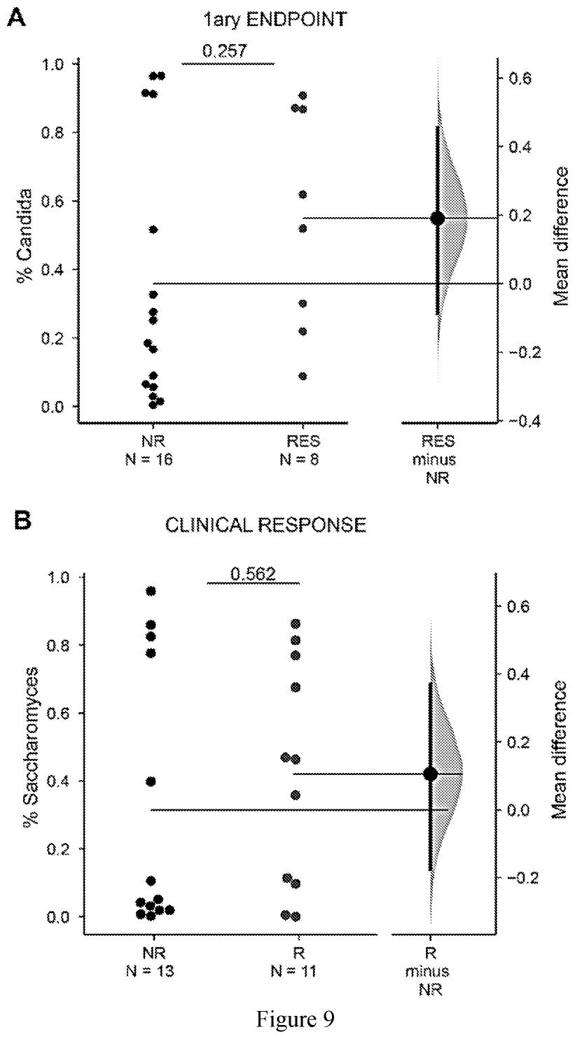

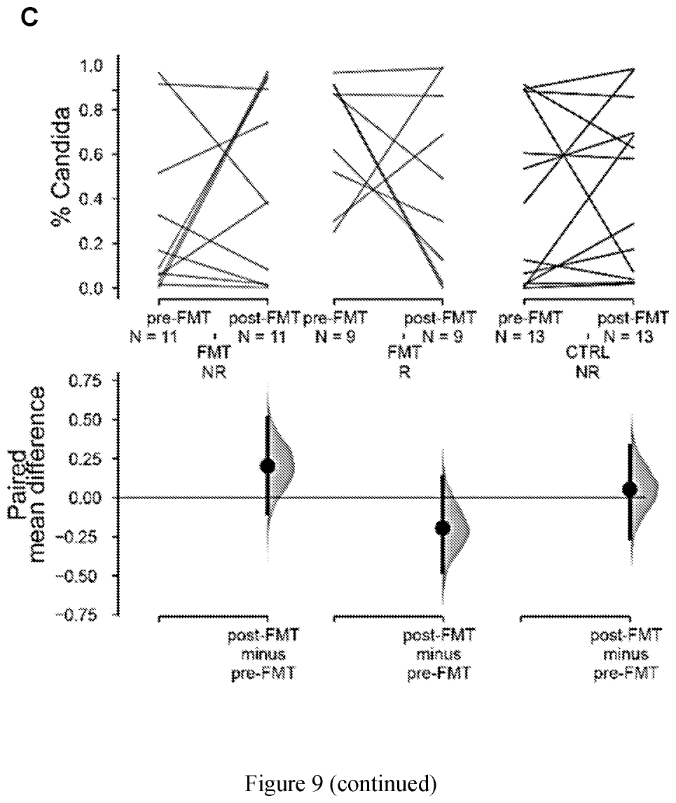

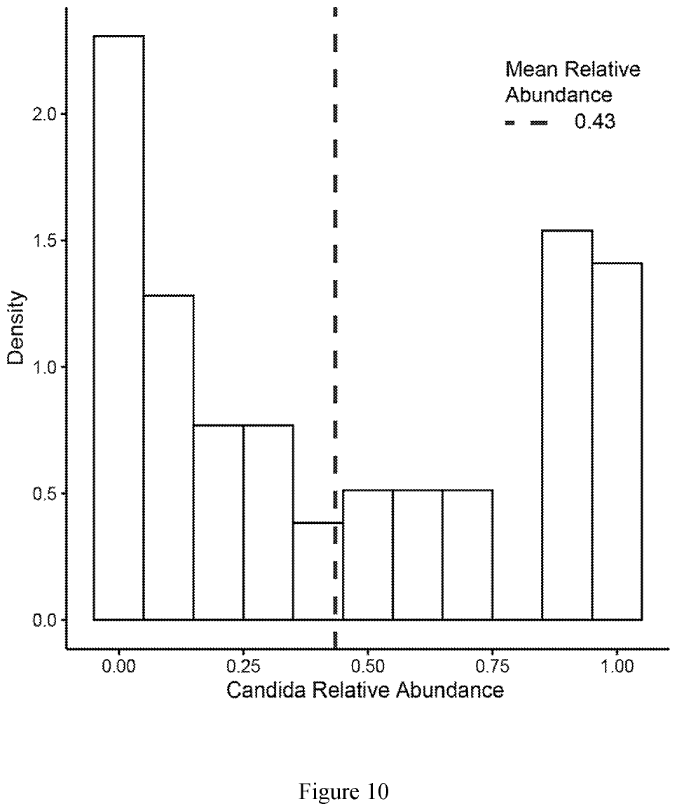

These findings were corroborated by a significant negative correlation between the pre-FMT relative abundance of Candida and the total Mayo Score post-FMT in the FMT recipients but not in the placebo controls (FIG. 2E). Similarly, higher levels of Candida were observed in endoscopic responders with respect to non-responders (FIG. 9A, B) and in patients achieving the primary endpoint (FIG. 9A), although the difference did not reach statistical significance.

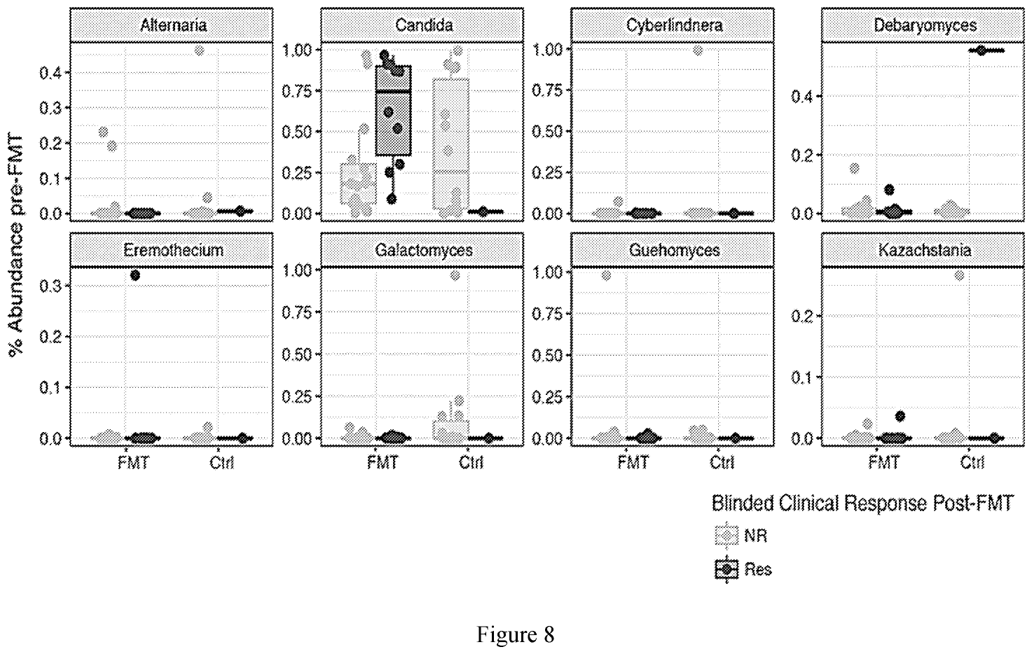

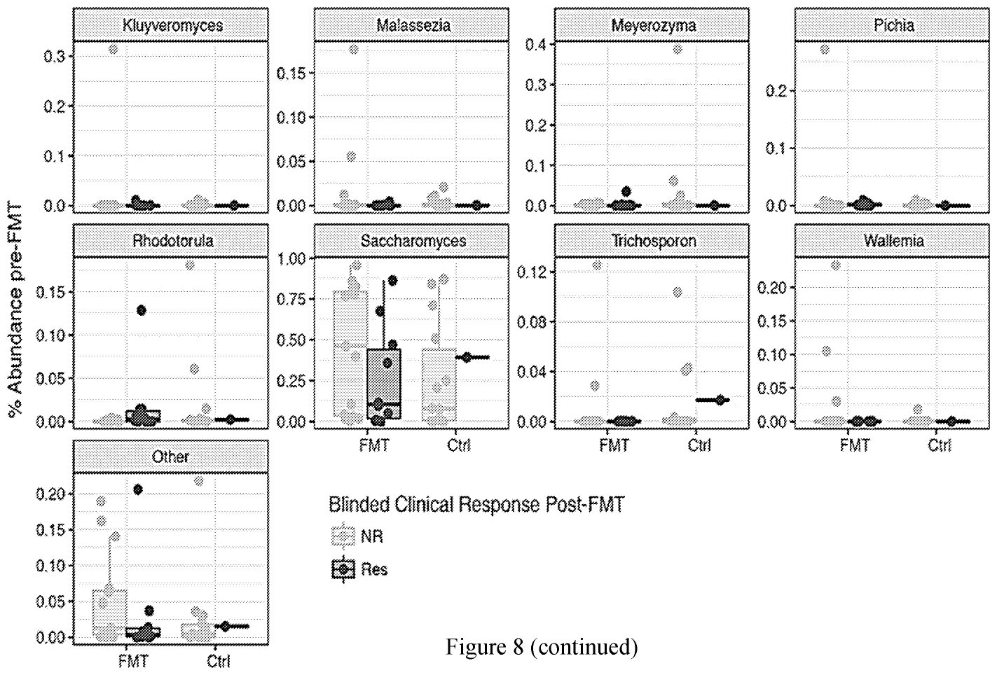

The pre-FMT difference between responders and non-responders was specific to Candida species as Saccharomyces and other less abundant genera were similar between the two groups (FIGS. 8, & 9C). Altogether, these findings suggest that preexisting intestinal Candida colonization is associated with positive clinical outcome of FMT.

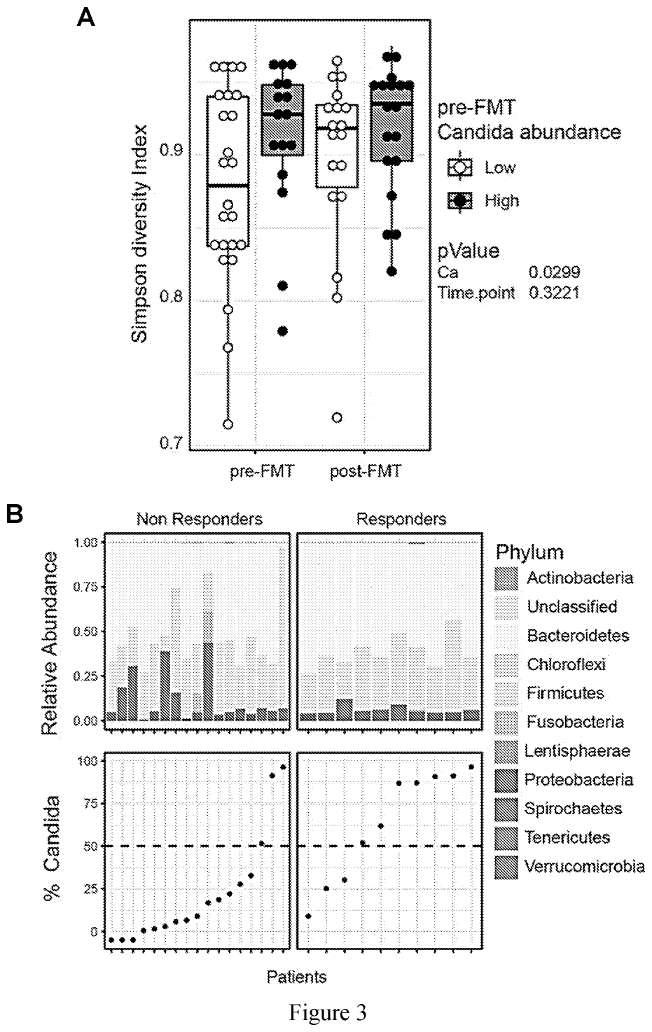

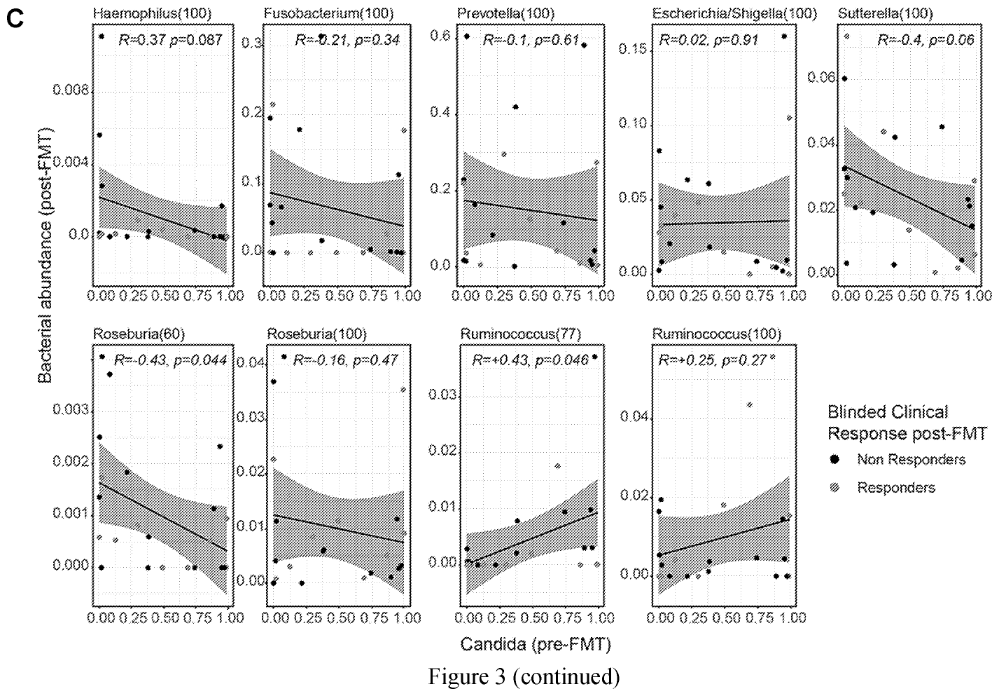

We investigated if in clinical responders, FMT might reduce the intestinal burden of Candida, thus attenuating its pro-inflammatory effects, or alternatively, Candida might directly affect bacterial expansion or engraftment and thus influence the outcome of FMT. To investigate the possible interaction between Candida species and the bacterial microbiota, we analyzed the bacterial composition of the trial participants stratifying pre-FMT subjects by Candida species abundance (FIG. 3A, B, FIG. 9D, 9E, 10). Notably, UC patients with high levels of Candida pre-FMT, had significantly higher bacterial alpha diversity that persisted at week 8 post-treatment irrespective of FMT treatment (FIG. 3A,B), suggesting that Candida might contribute to the establishment of a niche more permissive to FMT engraftment.

We next assessed whether bacteria found to be associated with FMT outcome would correlate with the initial abundance of Candida. It has been previously reported that, Haemophilus, Fusobacterium, Prevotella, and Sutterella genera correlated with a negative FMT outcome (Paramsothy et al. Gastroenterology 156 (5):1440-1454). Among these genera, both Haemophilus and Sutterella negatively correlated with the abundance of Candida (Pearson correlation, FIG. 3C). In contrast, among the genera previously reported to positively correlate with FMT outcome, we found that Roseburia negatively correlated with Candida abundance, whereas Ruminococcus displayed a significant positive correlation with Candida abundance. Our results suggest the existence of complex trans-kingdom interactions and highlight a possible mechanism in which Candida might affect the engraftment of particular bacterial strains.

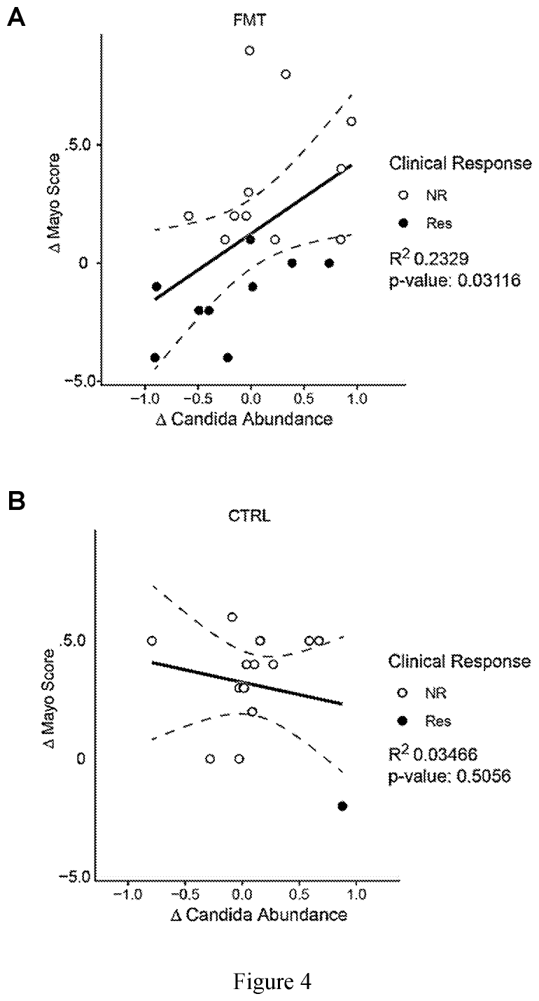

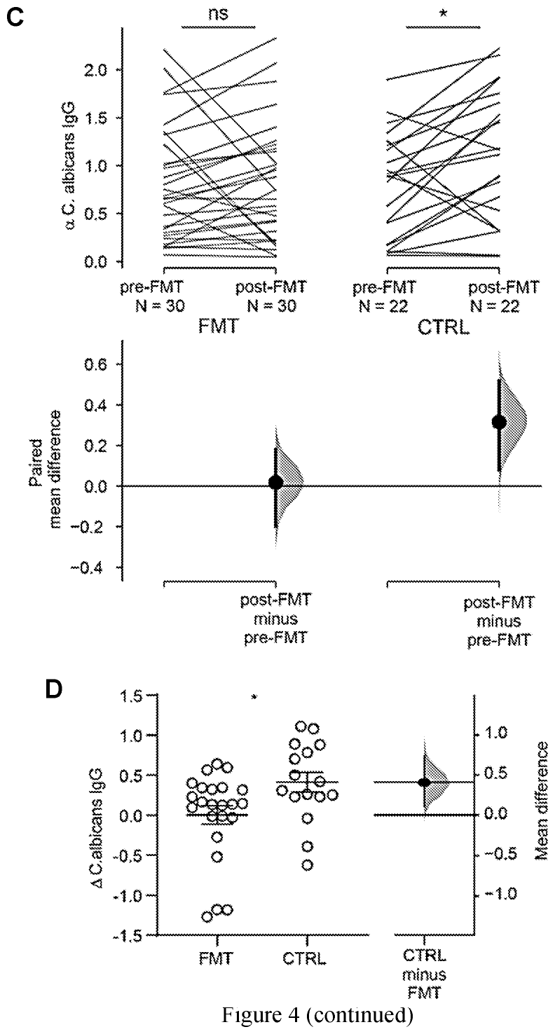

Candida spp. have been associated with intestinal inflammation and experimental studies have shown that several Candida species can worsen the outcome of intestinal inflammation in mouse models We thus assessed the changes in Candida abundance following treatment in FMT and placebo recipients. In FMT recipients the reduction in Candida abundance correlated positively with a reduction of endoscopic and clinical disease severity as indicated by change in Mayo score (FIG. 4A). This correlation was only observed in FMT recipients and not in placebo controls (FIG. 4B), suggesting that a reduction in disease severity does not cause the reduction of Candida abundance. These result indicate that FMT might act at least partially by reducing the abundance of Candida in the GI tract. In our cohort, Candida albicans was the most prevalent and abundant Candida species (FIG. 6F). We thus assessed the effect of FMT on the induction of immune response against Candida by assessing the levels anti-C. albicans IgG antibodies in the patents' serum. Over 8 weeks, placebo recipients experienced a significant increase in anti-Candida IgG responses (FIG. 4C-D). In contrast, anti-C. albicans IgG titers were stable in FMT recipients (FIG. 4C-D) during the same period. Collectively, these data suggest that FMT might act by reducing Candida abundance and containing pro-inflammatory immune responses induced by gut-fungi during intestinal inflammation.

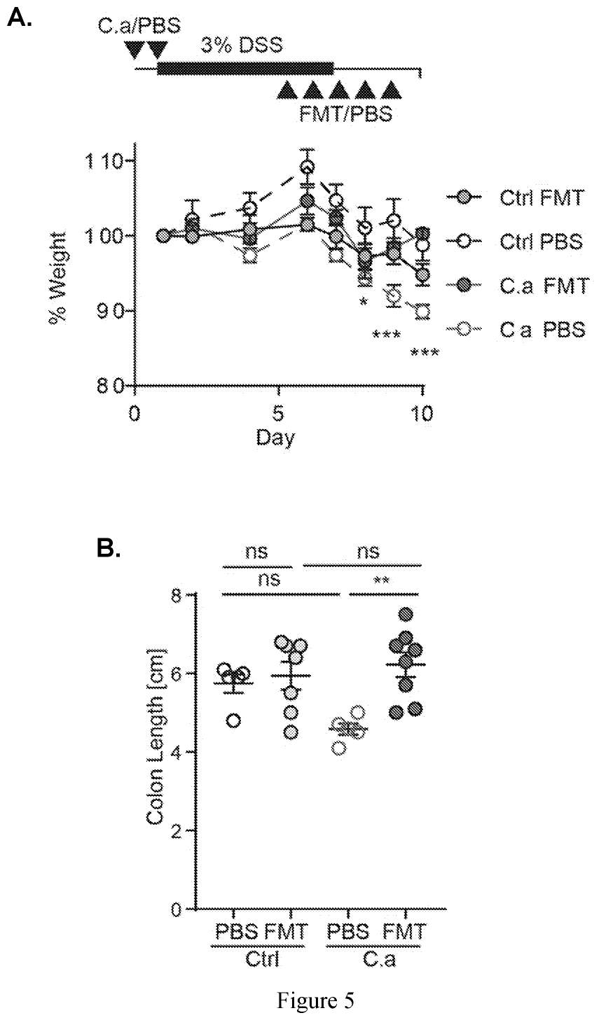

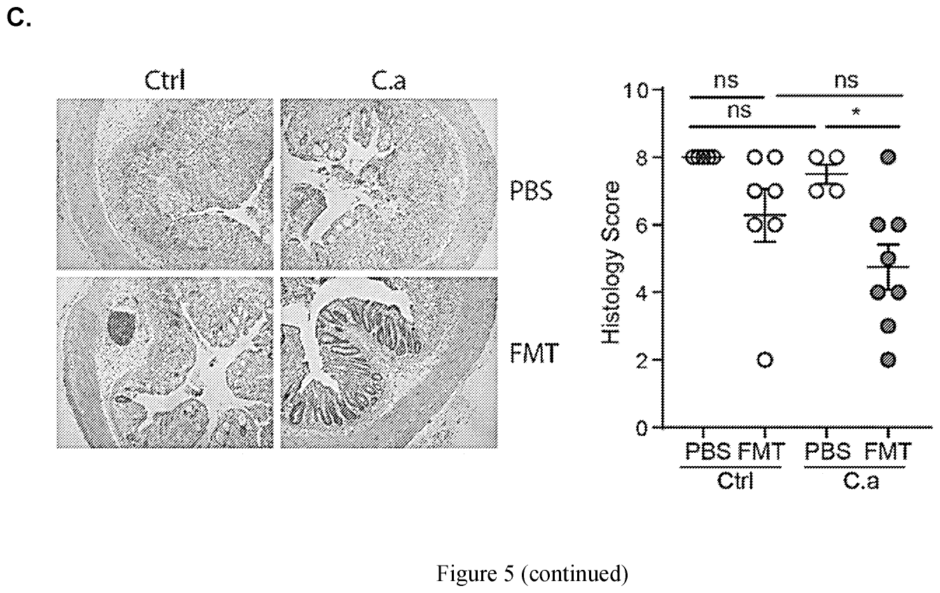

To model the engraftment of FMT in the colonic inflammation and dysbiosis occurring in people with increased intestinal permeability, which leads to detrimental effects on the host immune system and has been demonstrated in diseases such as inflammatory bowel disease (IBD), diabetes, asthma, mood disorders including depression, anxiety, autism, and colon cancer, we used the dextran sodium sulphate (DSS) induced chemical colitis model. In this model, C. albicans colonization exacerbated colitis in mice receiving the control PBS enemas that displayed severe weight loss and colon shortening (FIG. 5A-B). In contrast, mice subjected to FMT were protected from weight loss and colon shortening when compared with the C. albicans control group and mice subjected to FMT in absence of C. albicans colonization (FIG. 5A-B). Histological assessment of C. albicans mice subjected to FMT showed a reduced DSS induced damage in the distal colon when compared to the other treatment groups (FIG. 5C) thus suggesting that the presence of C. albicans promotes a positive response to FMT.

### DISCUSSION

The positive outcomes of several pilot- and larger-scale clinical studies highlight the increasing promise of FMT for the treatment of diseases with complex etiology such as IBD. Our mycobiota analysis of participants in the large, double blind, placebo-controlled FMT trial in UC, revealed a key role of trans-kingdom interactions between intestinal fungi and bacteria that positively affected the clinical outcome of FMT. Among the patients of the FOCUS cohort, we observed considerable heterogeneity in Candida abundance. Nonetheless, preexisting high Candida abundance in UC patients prior to FMT treatment was associated with increased bacterial diversity and a better therapy outcome. Our data suggest that a reduction of intestinal Candida colonization contributes to the positive outcome of FMT on intestinal inflammation.

While the present invention has been describes via various embodiments and examples, routine modifications will be apparent to those skilled in the art, and such modifications are intended to be within the scope of the present disclosure.

What is claimed is:

1. A method of treating an individual afflicted with an inflammatory bowel disease comprising:
a) in a sample obtained from the individual's gastrointestinal tract, determining the level of Candida sp. to be the same or higher than a reference level; and administering to the individual, a therapy comprising microbiota transplantation therapy (MTT), or
b) in a sample obtained from the individual's gastrointestinal tract, determining the level of Candida sp. to be lower than a reference level, administering a composition to the individual to increase the level of Candida sp. such that the Candida sp. levels increase to the same or higher level than the reference level, and administering to the individual, a therapy comprising MTT.

2. The method of claim 1, wherein the inflammatory bowel disease is ulcerative colitis or Crohn's disease.

3. The method of claim 1, wherein the sample from the individual's gastrointestinal tract is a fecal sample, anal swab, intestinal biopsy, fecal washing, or mucosal washing.

4. The method of claim 1, wherein the level of Candida sp. is determined by RT-qPCR, deep sequencing, or antibody-based assay.

5. The method of claim 1, wherein the reference level is an average from a plurality of healthy individuals who do not have inflammatory bowel disease.

6. The method of claim 1, wherein the reference level is an average from a plurality of individuals who previously had inflammatory bowel disease and responded positively to MTT.

7. The method of claim 1, wherein the MTT is carried out by colonoscopy, endoscopy, sigmoidoscopy, or by enema.

8. The method of claim 1, wherein MTT is administered in solid, powdered, or liquid form, or a combination thereof.

9. The method of claim 1, wherein in b), the Candida enhancing composition is administered for at least 1 to 10 days prior to MTT administration.

10. The method of claim 1, wherein the MTT is administered over a period of at least 2 weeks.

11. The method of claim 10, wherein the MTT is administered over a period of at least 8 weeks.

12. A kit for treating an inflammatory bowel disease, the kit comprising:
a) one or more agents for determining the level of Candida in a sample from the gastrointestinal tract of an individual;
b) a microbiota transplantation therapy (MTT) composition; and
optionally, a reference Candida level in the gastrointestinal tract indicating a cut-off at or above which level, the MTT should be administered.

13. The kit of claim 12, wherein the one or more agents for determining the level of Candida in a) comprise one or more of primers, and/or antibodies specific to Candida.

14. The kit of claim 12, wherein the MTT composition is in a solid, liquid, semi-solid, powdered form or combinations thereof, or enema form.
